(19) United States
(12) Patent Application Publication
(10) Pub. No.: US 2020/0030791 A1
(43) Pub. Date: Jan. 30, 2020

(54) MULTIMODE MICROFLUIDIC DATA ROUTING

(71) Applicant: Hewlett-Packard Development Company, L.P., Houston, TX (US)

(72) Inventors: Shameed Sait M.A., Bangalore (IN); Manish Giri, Corvallis, OR (US)

(21) Appl. No.: 16/095,371

(22) PCT Filed: May 31, 2016
(86) PCT No.: PCT/US2016/035110

§ 371 Date: Oct. 21, 2018

(30) Foreign Application Priority Data
Apr. 21, 2016 (IN) 201641013938

(51) Int. Cl. B01L 3/00; A61B 5/026

(52) U.S. Cl. CPC B01L 3/5027; A61B 5/026; B01L 2300/023; B01L 2200/147; B01L 2300/0627; B01L 2200/10

(57) ABSTRACT

A method for handling multimode microfluidic data may output first signals from a first sensor on a microfluidic chip, may output second signals from a second sensor on the microfluidic chip and may output a single data stream from the microfluidic chip. The single data stream may include the first signals from the first sensor and the second signals from the second sensor. The method may further receive the single data stream from the microfluidic chip, route the first signals from the first sensor to a first data processing thread and route the second signals from the second sensor to a second data processing thread.

### BACKGROUND

Various sensing devices are currently available for sensing different attributes of fluid, such as blood as an example. Such sensing devices often include a microfluidic chip having a sensor dedicated to sensing a particular attribute of the fluid.

### BRIEF DESCRIPTION OF THE DRAWINGS

FIG. 1 is a schematic diagram of an example microfluidic chip.

FIG. 2 is a flow diagram of an example method for handling multi-sensor data on a single microfluidic chip

FIG. 3 is a schematic diagram of an example fluid testing system comprising the example microfluidic chip of FIG. 1.

FIG. 4 is a flow diagram of an example method for handling multimode sensor data.

FIG. 5 is a schematic diagram of an example mobile analyzer.

FIG. 6 is a flow diagram of an example method for processing multimode sensor data.

FIG. 7 is a schematic diagram of another example fluid testing system.

FIG. 8 is a schematic diagram of another example fluid testing system.

FIG. 9 is a top view of an example cassette board supporting an example microfluidic chip and funnel.

FIG. 10 is a bottom view of the example cassette board of FIG. 9.

FIG. 11 is a fragmentary sectional view of a portion of the cassette board of FIG. 9.

FIG. 12 is a top view of an example microfluidic chip of an example cassette of the fluid testing system of FIG. 8.

FIG. 13 is an enlarged fragmentary view of a portion of the microfluidic chip of FIG. 12.

FIG. 14 is a diagram illustrating an example process of handling multimode sensor data.

### DETAILED DESCRIPTION OF EXAMPLES

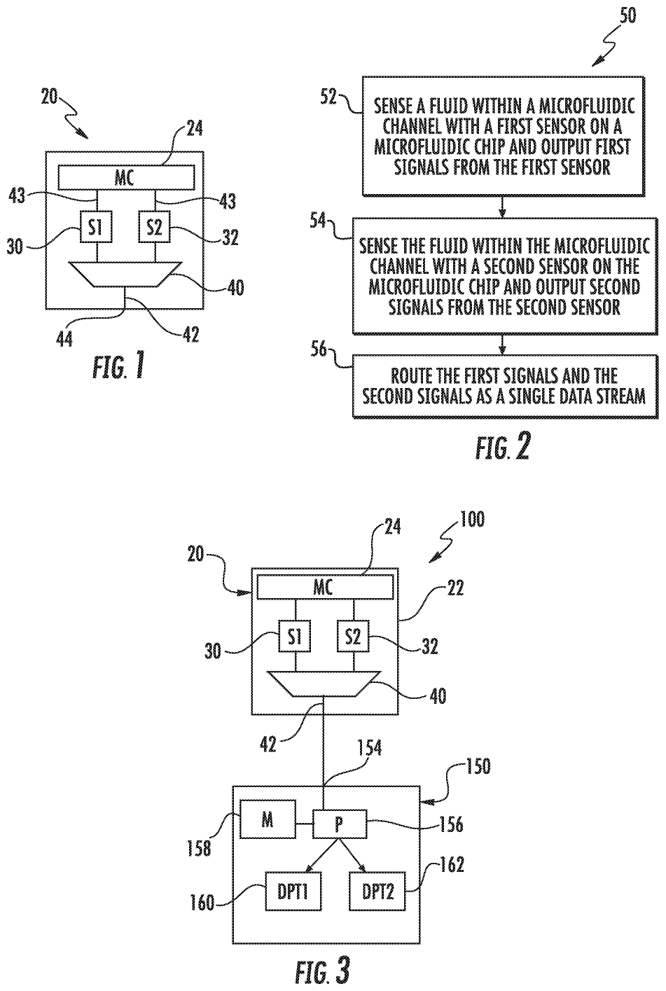

FIG. 1 schematically illustrates an example microfluidic chip 20. As will be described hereafter, microfluidic chip routes signals from multiple sensors through or as part of a single data stream. As a result, valuable chip real estate and communication bandwidth may be conserved, facilitating the use of microfluidic chip 20 as part of a fluid testing system that is compact, low-cost and mobile.

Microfluidic chip 20 may be used as part of a larger fluid testing system in which characteristics of fluids are tested for analysis. In one implementation, microfluidic chip 20 may be utilized in conjunction with a mobile analyzer. In some implementations, microfluidic chip 20 may be supported by an underlying larger board and/or housed by an outer body. In one implementation, microfluidic chip 20 may be provided as part of a cartridge or cassette which is connected directly or indirectly to a separate mobile analyzer that receives and utilizes signals from microfluidic chip 20.

Microfluidic chip 20 comprises substrate 22, microfluidic channel 24, sensors 30, 32, multiplexer 40 and data line 42. Substrate 22 comprises a platform that supports channel 24, sensors 30, 32, multiplexer 40, and data line 42 as well as other components and circuitry of microfluidic chip 20, such as electrically conductive traces, integrated circuits (such as field programmable gate arrays and application-specific integrated circuits) as well as other electronics. In one implementation, substrate 22 may be formed from silicon or silicon based materials. In another implementation, substrate 22 may be formed from a polymer or other materials.

Microfluidic channel 24 comprises a fluid passage formed in and/or along a surface of substrate 22 through which fluid being tested may flow or be circulated. In one implementation, microfluidic channel 24 receives fluid through an input port and funnel. Microfluidic channel 24 guides the flow of fluid being tested to different sensing regions where sensors 30, 32 detect characteristics of the fluid. In one implementation, microfluidic channel 24 has a continuous uniform cross-sectional area or a varying cross-sectional area of less than 1000 μm².

In one implementation, microfluidic channel 24 may comprise constrictions having cross-sectional areas similar to the size of a single cell or analyte particle so as to restrict the number of cells or particles that may flow across or relative to one of sensors 30, 32 in parallel. In one implementation, such constrictions may be dimensioned so as to facilitate single file flow of cells or other analyte carried within the fluid being tested. In some implementations, the constrictions are provided by narrowing sides of microfluidic channel 24. In other implementations, the constricted are provided by spaced pillars or columns between the sides of microfluidic channel 24. In one implementation in which the cells being tested have a general or average maximum data mention of 6 μm, such constrictions may have a cross-sectional area of 100 μm², with a length of 10 μm a, width of 10 μm and a height of 10 μm. Although schematically illustrated as being linear, in some implementations, microfluidic channel 24 may be serpentine, may be curved or may have U-shape, branching off of a central passage or slot. In one implementation, such constrictions have a width of no greater than 30 μm.

Sensors 30 and 32 comprise devices supported by substrate 22 and connected to or proximate to microfluidic channel 24 (as indicated by the schematic lines 43) so as to sense and output signals indicating characteristics of the fluid (and/or any particles or cells carried within the fluid). In one implementation, sensors 30, 32 comprise identical sensors at different locations along microfluidic channel 24. In another implementation, sensors 30, 32 may be at the same general location along microfluidic channel 24 or may be at different locations along microfluidic channel 24, wherein sensors 30, 32 are of a single type but have different performance characteristics, such as different levels of sensitivity, signal output and the like. In yet another implementation, sensors 30, 32 may comprise different types of sensors, sensors that detect different physical characteristics of the fluid and/or cells/particles carried within the fluid. For example, in one implementation, sensors 30, 32 may comprise different sensors selected from a group of sensors consisting of electrical impedance sensors, optical sensors, thermal or temperature sensors, electrochemical sensors and pressure sensors.

Multiplexer 40 comprises electronic circuitry, such as a multiple input, a single output switch, that routes signals from both of sensors 30 and 32 as a single data stream along data line 42 to an output point 44 of microfluidic chip 20. In one implementation, output point 44 may comprise an electrical contact pad. In another implementation, output point may be part of a continuous electrically conductive trace that continues beyond the edge of substrate 22 to an electrical connector supported by a board or other platform supporting microfluidic chip 20. In one implementation, multiplexer 40 routes signals from sensors 30, 32 to data line 42 in an alternating equal fashion. In another implementation, multiplexer 40 routes signals from sensors 30, 32 to data line 42 as such signals are received, wherein a priority may be given to signals from one of sensors 30, 32 over the other of sensors 30, 32. Because multiplexer 40 routes signals from multiple sensors 30, 32 through or as part of a single data stream along data line 42, valuable chip real estate and communication bandwidth is conserved, facilitating the use of microfluidic chip 20 as part of a fluid testing system that is compact, low-cost and mobile.

FIG. 2 is a flow diagram of an example method 50 for handling multi-sensor data on a single microfluidic chip, such as microfluidic chip 20. Although method 50 is described with respect to microfluidic chip 20, method 50 may be carried out with any of the microfluidic chip described hereafter or similarly constructed microfluidic chips.

As indicated by block 52, sensor 30 senses a fluid within microfluidic channel 24 on microfluidic chip 20 and outputs first signals. As indicated by block 54, sensor 32 senses the fluid within the microfluidic channel 24 on microfluidic chip 20 and output second signals. As indicated by block 56, multiplexer 40 routes the first signals and the second signals as a single data stream. . Because method 50 routes signals from multiple sensors 30, 32 through or as part of a single data stream along data line 42, valuable chip real estate and communication bandwidth is conserved, facilitating the use of microfluidic chip 20 as part of a fluid testing system that is compact, low-cost and mobile.

FIG. 3 schematically illustrates an example fluid testing system 100 comprising microfluidic chip 20. In addition to microfluidic chip 20, fluid testing system 100 comprises mobile analyzer 150 for analyzing fluid samples, such as blood samples, received from microfluidic chip 20. As will be described hereafter, mobile analyzer 150 provides a portable platform for analyzing a stream of signals or data in real time as signals are received from the microfluidic chip 20. In the example illustrated, mobile analyzer 150 utilizes multi-threading in a way such that the mobile analyzer 150 is able to process the large amounts of data continuously received from the ongoing fluid tests and is able to output results of the data analysis in a timely fashion.

Mobile analyzer 150 comprises a mobile or portable electronic device or self-contained unit. In one implementation, mobile analyzer 150 comprises a computing device that is sized and weighted to be manually held by one person of a user for prolonged periods of time during its use such as during the actual testing in receipt of data, during the analysis of the data and presentation of the results. In one implementation, mobile analyzer 150 comprises a computing device embodied in a single rectangular or substantially rectangular panel (substantially meaning that the corners may be rounded, cropped or cut off), wherein a majority of a face of the single rectangular panel comprises a touch screen serving as both a display and an input. For example, in one implementation, mobile analyzer 150 comprises a tablet computer that has a diagonal corner-two-corner dimension of less than or equal to 12 inches (nominally a height of less than 8 to 10 inches and a width of less than or equal to 7 inches), a thickness of less than or equal to 0.4 inches, and a weight of less than or equal to 1.5 pounds and nominally less than or equal to 1 pound. In still other implementations, mobile analyzer 150 comprises a smart phone, flash player or phablet having a length less than or equal to 7 inches, a width of less than or equal to 4 inches, a thickness of less than or equal to 0.5 inches and a weight of less than or equal to 8 ounces.

In another implementation, the mobile analyzer 150 comprises a self-contained computing device that is sized and weighed to be manually carried from one testing place to another testing place by a single person between uses, wherein the computing device is placed upon a supporting surface during the actual testing, data analysis and results presentation. For example, in one implementation, mobile analyzer 150 comprises a king-sized tablet computer, sometimes referred to as a tabletop or multi-mode computer having a diagonal corner-to-corner dimension of less than or equal to 21 inches, a thickness of less than or equal to 1 inch and a weight of less than or equal to 10 pounds. In yet other implementations, mobile analyzer 150 comprises a laptop or notebook computing device.

As schematically shown FIG. 1, mobile analyzer 150 comprises housing 152, data input 154, processor 156 and memory 158. Housing 152 (schematically illustrated) houses electronics and componentry of mobile analyzer 150. Data input 154 comprises an electrical connector that facilitates communication between mobile analyzer 150 and data line 42 of microfluidic chip 20 such that mobile analyzer 150 receives a single stream of data including signals from both sensors 30 and 32 of microfluidic chip 20. In one implementation, data input 154 comprises an electrical connection port to receive a plug. In another implementation, data input 154 comprises an electrical plug or an electrical plug connected to a cord extending from housing 152. In one implementation, data input 154 comprises a universal serial bus port. In one implementation, data input 154 facilitates direct electrical connection and communication between mobile analyzer 150 and microfluidic chip 20. In yet other implementations, data input 154 may facilitate direct electrical connection of mobile analyzer 150 to an intermediate electronic device that indirectly connects mobile analyzer 150 to data line 42 of microfluidic chip 20.

Processor (P) 156 comprises electronics such as a processing unit that receives the single data stream comprising signals from sensors 30, 32 and that processes such signals. For purposes of this application, the term “processing unit” shall mean a presently developed or future developed electronics or processing hardware that executes sequences of instructions contained in a non-transitory memory, such as memory 158. Execution of the sequences of instructions causes the processing unit to perform steps such as generating control signals. The instructions may be loaded in a random access memory (RAM) for execution by the processing unit from a read only memory (ROM), a mass storage device, or some other persistent storage. In other embodiments, hard wired circuitry may be used in place of or in combination with software instructions to implement the functions described. For example, processor 156 may be embodied as part of one or more application-specific integrated circuits (ASICs). Unless otherwise specifically noted, the controller is not limited to any specific combination of hardware circuitry and software, nor to any particular source for the instructions executed by the processing unit.

In the example illustrated, memory (M) 160 comprises computer-readable instructions or programming that direct processor 156 to identify different types of data represented by different signals contained in the single data stream received from microfluidic chip 20 across data line 42. Instructions in memory 158 direct processor 156 to discern, distinguish and identify (A) data and signals originating from or based upon signals output by sensor 30 in the single data stream from (B) data and signals originating from or based upon signals output by sensor 32 in the single data stream. Instructions in memory 158 direct processor 156 to route the signals and/or data originating from or based upon signals from sensor 30 for being subsequently analyzed or processed by data processing thread (DPT1) 160. Instructions in memory 158 direct processor 156 to route the signals and/or data originating from or based upon signals from sensor 32 for being subsequently analyzed or processed by data processing thread (DPT2) 162. In one implementation, such instructions route the differing data signals to different queues or buffers for subsequent processing by data processing threads 160, 162.

Instructions in memory 158 further direct processor 156 to analyze the different data or different signals in the different data processing threads 160, 162. In the example illustrated, processor 156 concurrently carries out both data processing threads 160, 162, concurrently processing and analyzing signals from both sensors 30, 32 on microfluidic chip 20. As a result, mobile analyzer 150 may concurrently present the results of output from each of data processing threads 160, 162 in real time as such signals are received in the single data stream. In one implementation, the output is displayed on a display screen. In one implementation, the output is continuously updated and plotted on the display screen in the form of a graph.

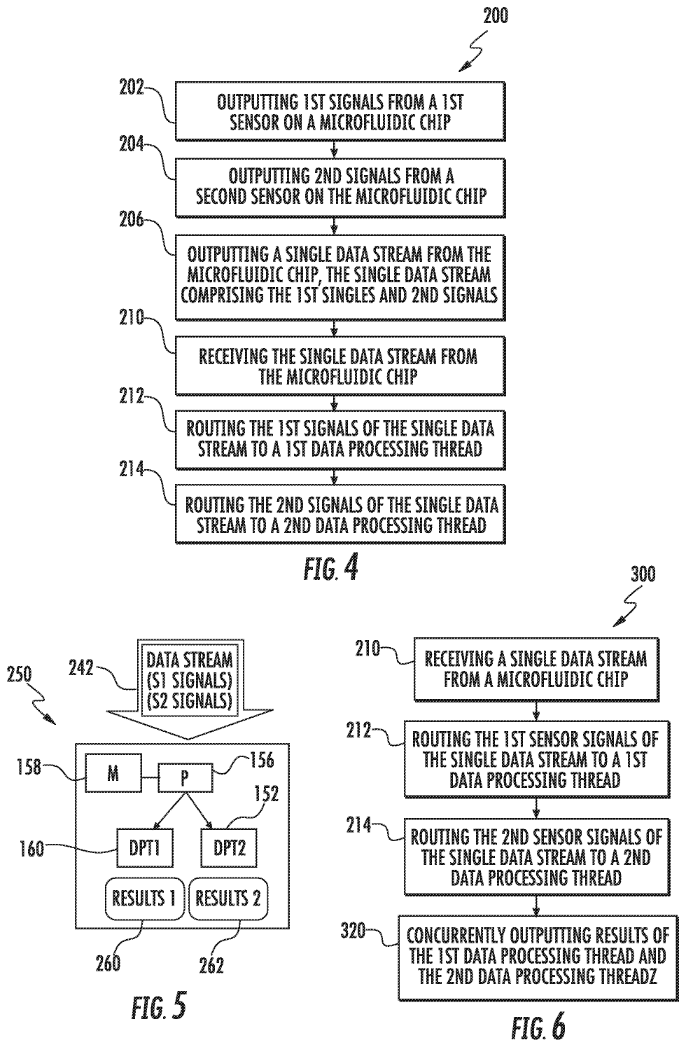

FIG. 4 is a flow diagram of an example method 200 for multi-sensor microfluidic data handling and analysis. For purposes of this disclosure, method 200 is described as being carried out by fluid sensing system 100. It should be understood that method 200 may alternatively be carried out by any of the fluid sensing systems described hereafter or other fluid sensing systems.

As indicated by block 202, sensor 30 of microfluidic chip 20 outputs first signals representing data pertaining to fluid within microfluidic channel 24. Similarly, as indicated by block 204, sensor 32 of microfluidic chip 20 outputs second signals representing data pertaining to fluid within microfluidic channel 24. As discussed above, in one implementation, sensors 30, 32 comprise identical sensors at different locations along microfluidic channel 24. In another implementation, sensors 30, 32 may be at the same general location along microfluidic channel 24 or may be at different locations along microfluidic channel 24, wherein sensors 30, 32 are of a single type but have different performance characteristics, such as different levels of sensitivity, signal output and the like. In yet another implementation, sensors 30, 32 may comprise different types of sensors, sensors that detect different physical characteristics of the fluid and/or cells/particles carried within the fluid. For example, in one implementation, sensors 30, 32 may comprise different sensors selected from a group of sensors consisting of electrical impedance sensors, optical sensors, thermal sensors, temperature sensors and pressure sensors.

In one implementation, such signals are concurrently output by sensors 30, 32 with the same frequency. In other implementations, sensors 30, 32 may output the respective signals at different frequencies. Such signals are transmitted to multiplexer 40.

As indicated by block 206, multiplexer 40 receives the signals from sensors 30 and 32 and outputs a single data stream from microfluidic chip 20 comprising both the first signals from sensor 30 and the second signals from sensor 32. In one implementation, multiplexer 40 routes signals from sensors 30, 32 to data line 42 in an alternating equal fashion. In another implementation, multiplexer 40 routes signals from sensors 30, 32 to data line 42 as such signals are received, wherein a priority may be given to signals from one of sensors 30, 32 over the other of sensors 30, 32. Because multiplexer 40 routes signals from multiple sensors 30, 32 through or as part of a single data stream along data line 42, valuable chip real estate and communication bandwidth is conserved, facilitating the use of microfluidic chip 20 as part of a fluid testing system that is compact, low-cost and mobile.

As indicated by block 210, mobile analyzer 150 receives the single data stream containing the signals from both sensors 30 and 32. As indicated by blocks 212 and 214, instructions in memory 158 direct processor 156 to discern, distinguish and identify (A) data and signals originating from or based upon signals output by sensor 30 in the single data stream from (B) data and signals originating from or based upon signals output by sensor 32 in the single data stream. As indicated by block 212, instructions in memory 158 direct processor 156 to route the signals and/or data originating from or based upon signals from sensor 30 for being subsequently analyzed or processed by data processing thread (DPT1) 160. As indicated by block 214, instructions in memory 158 direct processor 156 to route the signals and/or data originating from or based upon signals from sensor 32 for being subsequently analyzed or processed by data processing thread (DPT2) 162. In one implementation, such instructions route the differing data signals to different queues or buffers for subsequent processing by data processing threads 160, 162.

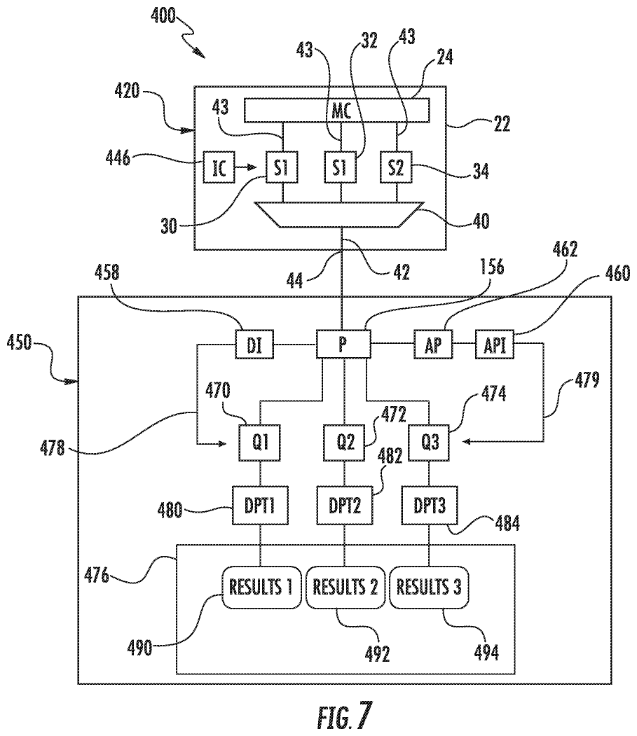

FIG. 5 schematically illustrates an example mobile analyzer 250 that may be utilized as part of system 100. Mobile analyzer 250 is similar to mobile analyzer 150 except that mobile analyzer 250 is specifically illustrated as concurrently outputting the results 260 and 262 of data processing threads 161 and 162, respectively. FIG. 6 is a flow diagram of an example method 300 for analyzing multi-mode or multi sensor data. Method 300 is described as being carried out by mobile analyzer 250 in FIG. 5. In other implementations, method 300 may be carried out by other similar mobile analyzers. For purposes of illustration, those components mobile analyzer 250 or those steps of method 300 which correspond to components of mobile analyzer 150 or steps of method 200, respectively, are numbered similarly.

As indicated by block 210 in FIG. 6 and as schematically illustrated in FIG. 5, mobile analyzer 250 receives a single data stream 242 from a microfluidic chip, such as microfluidic chip 20 described above. The single data stream 242 comprises first signals (S1) from a first sensor, such a sensor 30, and second signals (S2) from a second sensor, such a sensor 32, on the microfluidic chip.

As indicated by blocks 212 and 214, mobile analyzer 250 routes the first sensor signals of the single data stream to a first data processing thread 160. Mobile analyzer 250 routes second signals of the single data stream to a second data processing thread 162. Such routing carried out in blocks 212 and 214 is described above with respect to method 200. Such processing threads 160 and 162 are concurrently carried out by processor 156, following instructions contained in memory 158.

As indicated by block 320, mobile analyzer 250 concurrently outputs the results 260, 262 of data processing thread 160 and data processing thread 162, respectively. As a result, mobile analyzer 150 may concurrently present the results of output from each of data processing threads 160, 162 in real time as such signals are received in the single data stream. In one implementation, the results 260, 262 are displayed on a display screen. In one implementation, the results 260, 262 of both sensors 30, 32 are continuously updated and plotted on the display screen in the form of a graph.

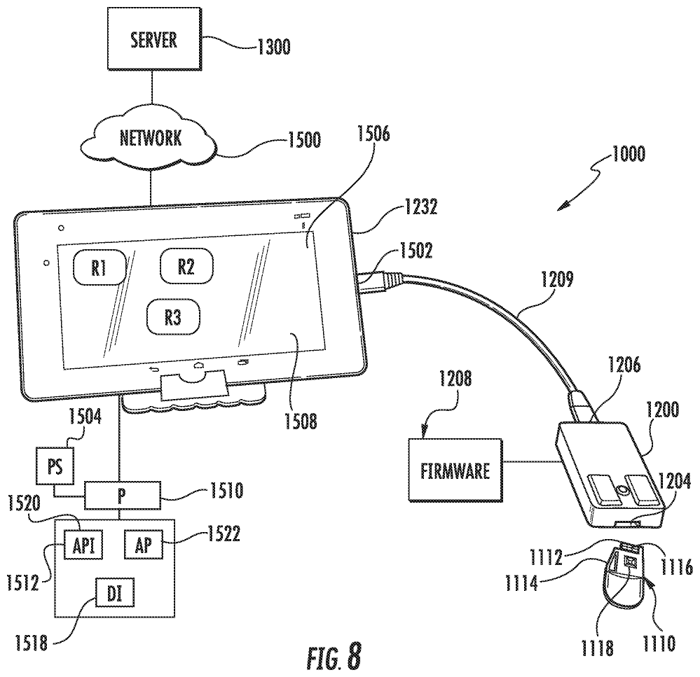

FIG. 7 schematically illustrates another example fluid testing system 400. Fluid testing system 400 comprises microfluidic chip 420 and mobile analyzer 450. Microfluidic chip 420 is similar to microfluidic chip 20 except that microfluidic chip 420 comprises three sensors, sensors 30, 32 and 34 and that microfluidic chip 20 additionally comprises integrated circuit 446. Those remaining components of microfluidic chip 420 which correspond to components of microfluidic chip 20 are numbered similarly.

Sensors 30 and 32 are described above with respect to fluidic chip 20. Sensor 34 comprises a device supported by substrate 22 and connected to or proximate to microfluidic channel 24 (as indicated by the schematic lines 43) so as to sense and output signals indicating characteristics of the fluid (and/or any particles or cells carried within the fluid). In one implementation, sensors 30, 32 and 34 comprise identical sensors at different locations along microfluidic channel 24. In another implementation, sensors 30, 32 and 34 may be at the same general location along microfluidic channel 24 or may be at different locations along microfluidic channel 24, wherein sensors 30, 32 and 34 are of a single type but have different performance characteristics, such as different levels of sensitivity, signal output and the like. In yet another implementation, sensors 30, 32 and 34 may comprise different types of sensors, sensors that detect different physical characteristics of the fluid and/or cells/particles carried within the fluid. For example, in one implementation, sensors 30, 32 and 34 may comprise different sensors selected from a group of sensors consisting of electrical impedance sensors, optical sensors, thermal or temperature sensors and pressure sensors. In one implementation, sensor 30 comprises an impedance sensor, sensor 32 comprises an optical sensor and sensor 34 comprises an optical sensor. Sensors 30, 32 and 34 outputs signals representing data, and the signals are transmitted to multiplexer 40. As with multiplexer 40 of microfluidic chip 20, multiplexer 40 of microfluidic chip 420 comprises electronic circuitry, such as a multiple input, a single output switch, that routes signals from each of sensors 30, 32 and 34 as a single data stream along data line 42 to an output point 44 of microfluidic chip 420.

Integrated circuit 446 comprises an application-specific integrated circuit or a field programmable gate array that controls the operation and output of sensors 30, 32 and 34. Integrated circuit 446 further controls the frequency or rate at which signals are output by sensors 30, 32 and 34. In one implementation, integrated circuit 446 controls the rate at which signals are output by sensors 30, 32 and 34 such that sensors 30, 32 and 34 output signals at different frequencies relative to one another. In one implementation in which sensor 30 comprises an impedance sensor, in which sensor 32 comprises an optical sensor and in which sensor 34 comprises a temperature or thermal sensor, integrated circuit 446 controls the output of such sensors such that sensor 30 outputs impedance data signals at a first frequency, such that sensor 32 output optical data signals at a second frequency, less than the first frequency, and such that sensor 34 outputs temperature or thermal data signals at a third frequency, less than the second frequency.

Mobile analyzer 450 is similar to mobile analyzer 150 described above except that mobile analyzer 450 is specifically illustrated as comprising data identification and routing instructions (DI) 458, application programming interface 460 and application program 462 as part of memory 158 (shown in FIG. 3). Mobile analyzer 450 further comprises queues 470 (Q1), 472 (Q2) and 474 (Q3) and display 476. Those remaining components of mobile analyzer 450 which correspond to components of mobile analyzer 150 are numbered similarly.

Data identification and routing instructions 458 comprise instructions contained in memory 158 that direct processor 156 to discern, distinguish and identify (A) data and signals originating from or based upon signals output by sensor 30 in the single data stream, (B) data and signals originating from or based upon signals output by sensor 32 in the single data stream and (C) data and signals originating from or based upon signals output by sensor 34 in the single data stream. In one implementation, data identifier instructions 458 direct processor 156 to read data bits contained in a header associated with each set or group of data bits from sensors 30, 32, 34, which are part of the single data stream from microfluidic chip 420. Data identification and routing instructions 458 direct processor 156 to route the signals and/or data originating from or based upon signals from sensor 30 to queue 470. Data identification instructions 458 direct processor 156 to route the signals and/or data originating from or based upon signals from sensor 32 to queue 472. Data identification and routing instructions 458 direct processor 156 to route the signals and/or data originating from or based upon signals from sensor 34 to queue 472. Queues 470, 472 and 474 comprise registers or buffers that temporarily store data for subsequent processing.

Application programming interface 460 may comprise a library of routines, protocols and tools, which serve as building blocks, for carrying out various functions or tests using signals from sensors 30, 32, 34 of microfluidic chip 22. Application programming interface 460 may comprise programmed logic that accesses the library and assembles the “building blocks” or modules to perform a selected one of various functions or tests using data from the different sensors 30, 32, 34 of microfluidic chip 420. For example, one application programming interface 460 may provide “building blocks” for performing cytology tests, coagulation tests and other tests.

Application programming interface 460 facilitates testing of fluids using signals from microfluidic chip 420 under the direction of different application programs. In other words, application programming interface 460 provides a universal programming or software set of commands for firmware that may be used by any of a variety of different application programs. For example, a user of mobile analyzer 450 is able to download or install any of a number of different application programs, wherein each of the different application programs is designed to utilize the application program interface 460 so as to carry out tests using cassette microfluidic chip 1130.

Application program 462 comprises overarching machine-readable instructions contained in memory 158 that facilitates user interaction with application programming interface 460. Application program 459 comprises software, code or instructions contained in the non-transitory memory 158 (shown in FIG. 3) that direct processor 156 to differently analyze the different sets of data in the different queues 470, 472 and 474 and originating from the different sensors 30, 32 and 34. As schematically indicated by arrow 479, application program 462 directs processor 156 to concurrently carry out three data processing threads 480, 482 and 484 using the data in queues 470, 472 and 474, respectively, as inputs.

Display 476 comprises a monitor, screen or LED display region that visibly presents the results 490, 492 and 494 of data processing threads 480, 482 and 484, respectively. The results 490, 492 and 494 are concurrently presented on display 476 in real time. In one implementation, the results 490, 492 and 494 based upon the data or signals from sensors 30, 32 and 34, respectively, are each continuously updated and plotted on the display screen in the form of a graph.

FIG. 8 schematically illustrates another example fluid diagnostic or testing system 1000. System 1000, portions of which are schematically illustrated, comprises microfluidic cassette 1110, cassette interface 1200, mobile analyzer 1232 and remote analyzer 1300. Overall, microfluidic cassette 1110 receives a fluid sample and outputs signals based upon sensed characteristics of the fluid sample. Interface 1200 serves as an intermediary between mobile analyzer 1232 and cassette 1110. Interface 1200 releasably connects to cassette 1110 and facilitates transmission of electrical power from mobile analyzer 1232 to cassette 1110 to operate pumps and sensors on cassette 1110. For purposes of this disclosure, the term “releasably” or “removably” with respect to an attachment or coupling of two structures means that the two structures may be repeatedly connected and disconnected to and from one another without material damage to either of the two structures or their functioning. Interface 1200 further facilitates control of the pumps and sensors on cassette 1110 by mobile analyzer 1232.

Mobile analyzer 1232 controls the operation of cassette 1110 through interface 1200 and receives data produced by cassette 1110 pertaining to the fluid sample being tested. Mobile analyzer 1232 analyzes data and produces output. Mobile analyzer 1232 further transmits processed data to remote analyzer 1300 for further more detailed analysis and processing. System 1000 provides a portable diagnostic platform for testing fluid samples, such as blood samples.

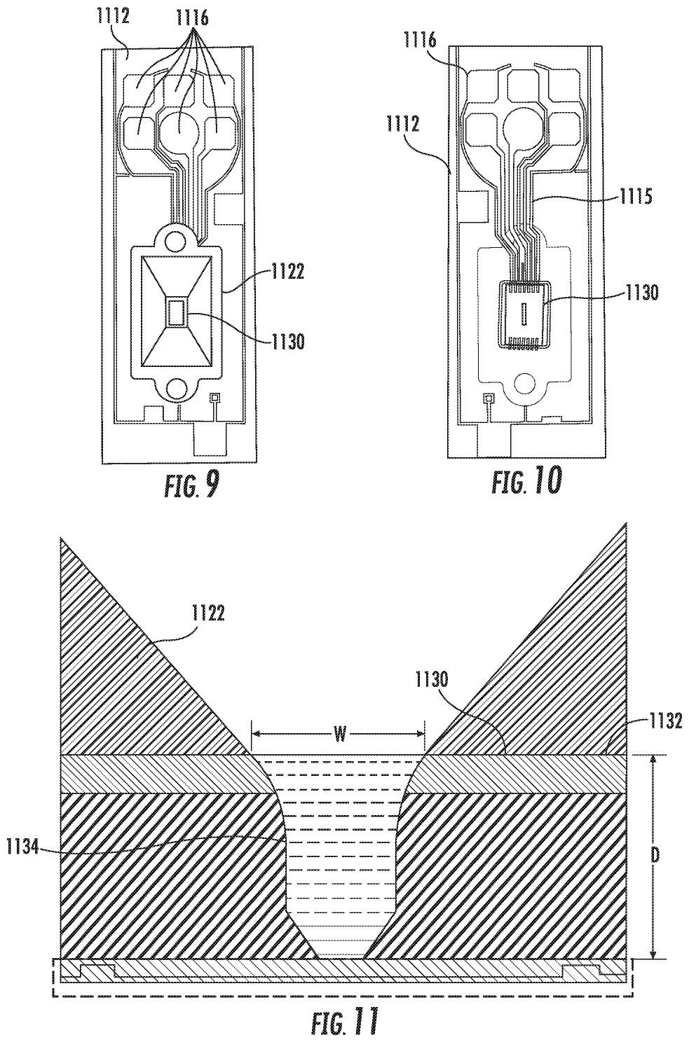

As shown by FIGS. 7-12, cassette 1110 comprises cassette board 1112, cassette body 1114 and microfluidic chip 1130. Cassette board 1112, shown in FIGS. 8 and 9, comprises a panel or platform in which or upon which fluid chip 1130 is mounted. Cassette board 1112 comprises electrically conductive lines or traces 1115 which extend from electrical connectors of the microfluidic chip 1130 to electrical connectors 1116 on an end portion of cassette board 1112. As shown in FIG. 8, electrical connectors 1116 are exposed on an exterior cassette body 1114 and are to be inserted into interface 1200 so as to be positioned in electrical contact with corresponding electrical connectors within interface 1200, providing electrical connection between microfluidic chip 1130 and cassette interface 1200.

Cassette body 1114 partially surrounds cassette board 1112 so as to cover and protect cassette board 1112 and microfluidic chip 1130. Cassette body 1114 facilitates manual manipulation of cassette 1110, facilitating manual positioning of cassette 1110 into releasable interconnection with interface 1200. Cassette body 1114 additionally positions and seals against a person's finger about a sample receiving port 1118 during the acquisition of a fluid or blood sample while directing the received fluid sample to microfluidic chip 1130 through a chip funnel 1122.

Sample receiving port 1118 comprises an opening into which a fluid sample, such as a blood sample, is to be received. Capillary action pulls in blood, from the finger, which forms the sample. In one implementation, the blood sample is of 5 to 10 microliters. Chip funnel 1122 comprises a funneling device that narrows down to chip 1130.

FIGS. 8, 9 and 10 illustrate microfluidic chip 1130. FIG. 9 illustrates a top side of cassette board 1112, chip funnel 1122 and microfluidic chip 1130. FIG. 9 illustrates microfluidic chip 1130 sandwiched between chip funnel 1122 and cassette board 1112. FIG. 10 illustrates a bottom side of the cassette board 1112 and microfluidic chip 1130. FIG. 11 is a cross-sectional view of microfluidic chip 1130 below chip funnel 1122. As shown by FIG. 11, microfluidic chip 1130 comprises a substrate 1132 formed from a material such as silicon. Microfluidic chip 1130 comprises a microfluidic reservoir 1134 formed in substrate 1132 and which extends below chip funnel 1122 to receive the fluid sample (with a reagent in some tests) into chip 1130. In the example illustrated, microfluidic reservoir has a mouth or top opening having a width W of less than 1 mm and nominally 0.5 mm. Reservoir 1030 has a depth D of between 0.5 mm and 1 mm and nominally 0.7 mm. As will be described hereafter, microfluidic chip 1130 comprises pumps and sensors along a bottom portion of chip 1130.

Although the Figures illustrate one specific example of microfluidic chip 1130, chip 1130 may comprise any suitable material, including silicon. The chip 1130 may contain different subcomponents, including sensors, pumps, and the like. The chip 1130 may have any suitable geometry, including shape and sizes. For example, the shape may be a parallelogram, such as a square, a rectangle, or any other shape. The shape may also be an irregular shape. The size of the chip need not be of any particular value. For example. In one example, the dimensions of the chip may be in the millimeter range. The term “dimensions” may refer to width, length, etc., depending on the shape of the chip. For example, the length of the chip may be between 0.5 mm and 10 mm—e.g., between 1 mm and 8 mm, between 2 mm and 6 mm, etc. Other values are also possible. In one example, the length is 2 mm. For example, the width of the chip may be between 0.1 mm and 5 mm—e.g., between 0.5 mm and 4 mm, between 1 mm and 2 mm, etc. Other values are also possible.

FIGS. 11 and 12 are enlarged views of microfluidic chip 1130. Microfluidic chip 1130 integrates each of the functions of fluid pumping, impedance sensing and temperature sensing on a low-power platform. As will be described hereafter, microfluidic chip 1133 recirculates portions of a fluid sample, which has been tested, back to an input or upstream side of the sensors of microfluidic chip 1133. As shown by FIG. 10, microfluidic chip 1130 comprises substrate 1132 in which is formed a microfluidic reservoir. In addition, microfluidic chip 1130 comprises multiple sensing regions 1135, each sensing region comprising a microfluidic channel 1136, micro-fabricated integrated sensors 1150, 1152, 1154, and a pump 1160.

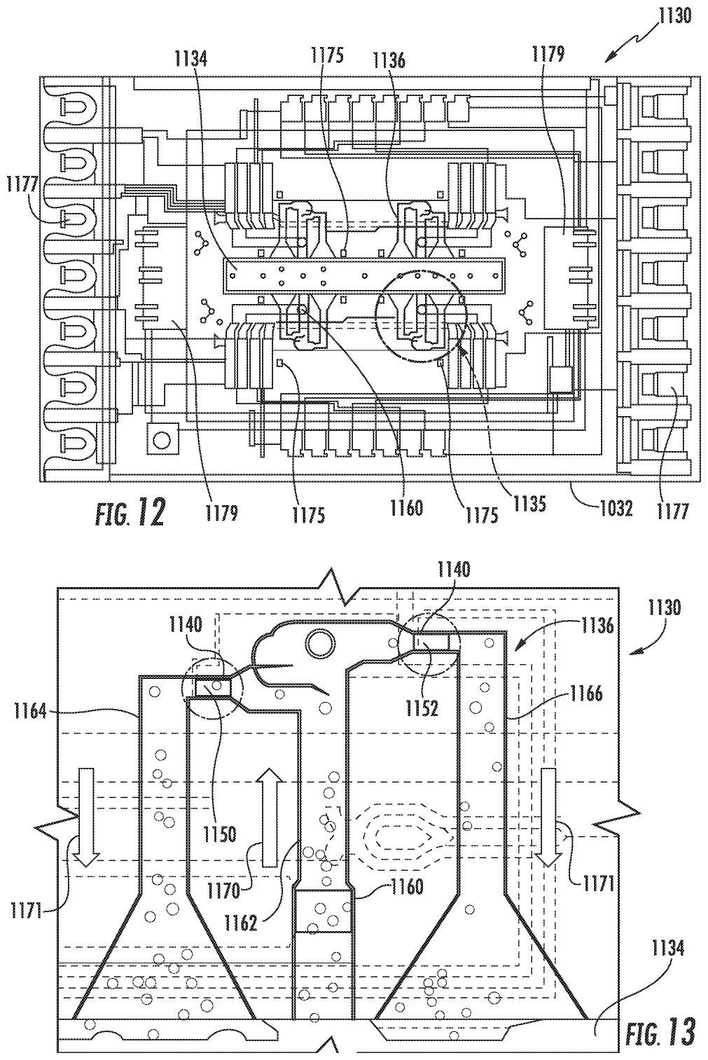

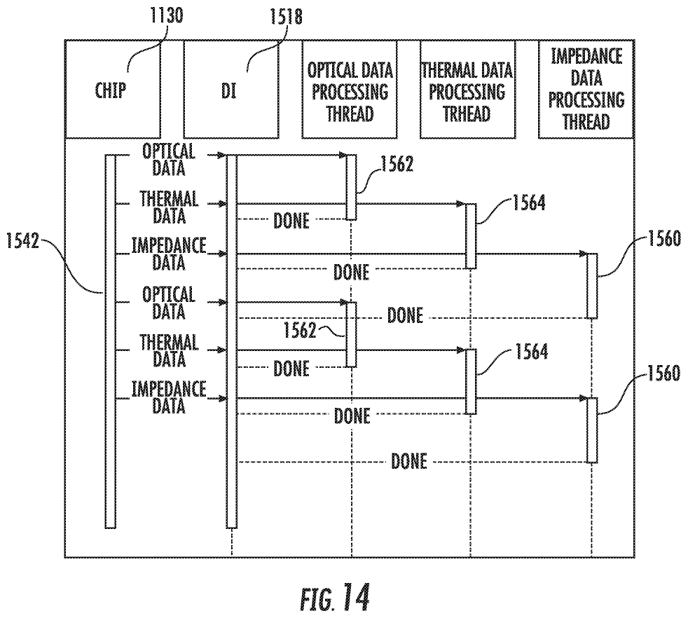

FIG. 13 is an enlarged view illustrating one of sensing regions 1135 of chip 1130 shown in FIG. 12. As shown by FIG. 13, microfluidic channel 1136 comprises a passage extending within or formed within substrate 1032 for the flow of a fluid sample. Channel 1136 comprises a pump containing central portion 1162 and a pair of sensor containing branch portions 1164, 1166. Each of branch portions 1164, 1166 comprises a funnel-shaped mouth that widens towards microfluidic reservoir 1134. Central portion 1162 extends from reservoir 1134 with a narrower mouth opening to reservoir 1134. Central portion 1162 contains pump 1160.

Sensor containing branch portions 1164, 1166 stem or branch off of opposite sides of central portion 162 and extend back to reservoir 1134. Each of branch portions 1164, 1166 comprises a narrowing portion, throat or constriction 1140 through with the fluid flows. In one implementation, branch portions 1164, 1166 are similar to one another. In another implementation, branch portions 1164, 1166 are shaped or dimensioned different from one another so as to facilitate different fluid flow characteristics. For example, the constrictions 1140 or other regions of portions 1164, 1166 may be differently sized such that particles or cells of a first size more readily flow through, if at all, through one of portions 1164, 1166 as compared to the other of portions 1164, 1166. Because portions 1164, 1166 diverge from opposite sides of central portion 1162, both of portions 1164, 1166 receive fluid directly from portion 1162 without fluid being siphoned to any other portions beforehand.

Micro-fabricated integrated sensors 1150, 1152 comprise micro-fabricated devices formed upon substrate 1032 within constrictions 1140. In one implementation, sensor 1150 comprises a micro-device that is designed to output electrical signals or cause changes in electrical signals that indicate properties, parameters or characteristics of the fluid and/or cells/particles of the fluid passing through constriction 1140. In one implementation, sensor 1150 comprises a cell/particle sensor that detects properties of cells or particles contained in a fluid and/or that detects the number of cells or particles in fluid passing across sensor 1138. For example, in one implementation, sensor 1150 comprises an electric sensor which outputs signals based upon changes in electrical impedance brought about by differently sized particles or cells flowing through constriction 1140 and impacting impedance of the electrical field across or within constriction 1140. In one implementation, sensor 1150 comprises a high side electrically charged electrode and a low side electrode formed within or integrated within a surface of channel 1136 within constriction 40. In one implementation, the low side electrode that is electrically grounded. In another implementation, the low side electrode is floating.

In one implementation, sensor 1152 comprises a microfabricated integrated optical sensor. For example, in one implementation, sensor 1152 comprises a silicon CMOS based optical sensor which can detect various wavelength and produce a corresponding electrical signal (voltage) which is then routed to the PCB/FPGA and then the data stream. In some implementations, sensor 1152 may comprise multiple CMOS sensors on the chip.

Pump 1160 comprises a device to move fluid through microfluidic channel 1136 and through constrictions 1140 across one of sensors 1150, 1152. Pump 1160 draws fluid from microfluidic reservoir 1134 into channel 1136. Pump 1160 further circulates fluid that has passed through constriction 1140 and across sensor 1150, 1152 back to reservoir 1134.

In the example illustrated, pump 1160 comprises a resistor actuatable to either of a pumping state or a temperature regulating state. The resistor of pump 1160 is formed from electrically resistive materials that are capable of emitting a sufficient amount of heat so as to heat adjacent fluid to a temperature above a nucleation energy of the fluid. The resistor is further capable of emitting lower quantities of heat so as to heat fluid adjacent the resistor of pump 1160 to a temperature below a nucleation energy of the fluid such that the fluid is heated to a higher temperature without being vaporized.

When the resistor forming pump 1160 is in the pumping state, pulses of electrical current passing through the resistor cause resistor to produce heat, heating adjacent fluid to a temperature above a nucleation energy of the adjacent fluid to create a vapor bubble which forcefully expels fluid across constrictions 1140 and back into reservoir 1134. Upon collapse of the bubble, negative pressure draws fluid from microfluidic reservoir 1134 into channel 1136 to occupy the prior volume of the collapsed bubble.

When the resistor forming pump 1160 is in the temperature regulating state or fluid heating state, the temperature of adjacent fluid rises to a first temperature below a nucleation energy of the fluid and then maintains or adjusts the operational state such that the temperature of the adjacent fluid is maintained constant or constantly within a predefined range of temperatures that is below the nucleation energy. In contrast, when resistor of pump 1160 is being actuated to a pumping state, the resistor of pump 1160 is in an operational state such that the temperature of fluid adjacent the resistor of pump 1160 is not maintained at a constant temperature or constantly within a predefined range of temperatures (both rising and falling within the predefined range of temperatures), but rapidly and continuously increases or ramps up to a temperature above the nucleation energy of the fluid.

In yet other implementations, pump 1160 may comprise other pumping devices. For example, in other implementations, pump 1160 may comprise a piezo-resistive device that changes shape or vibrates in response to applied electrical current to move a diaphragm to thereby move adjacent fluid across constrictions 1140 and back to reservoir 1134. In yet other implementations, pump 1160 may comprise other microfluidic pumping devices in fluid communication with microfluidic channel 1136. For example, in other implementations, pump 1160 may comprise an inertial pump, capillary pump, or a pneumatic pump.

As indicated by arrows in FIG. 13, actuation of pump 1160 to the fluid pumping state moves the fluid sample through central portion 1162 in the direction indicated by arrow 1170. The fluid sample flows through constrictions 1140 and across sensors 1138, where the cells within the fluid sample impact the electric field and wherein the impedance is measured or detected to identify a characteristic of such cells or particles and/or to count the number of cells flowing across the sensing volume of sensor 1138 during a particular interval of time. After passing through constrictions 1140, portions of the fluid sample continue to flow back to microfluidic reservoir 1134 as indicated by arrows 1171.

As further shown by FIG. 12, microfluidic chip 1130 additionally comprises temperature sensors 1175, electrical contact pads 1177 and multiplexer circuitry 1179. Temperature sensors 1175 are located at various locations amongst the sensing regions 1135. Each of temperature sensors 1175 comprises a temperature sensing device to directly or indirectly output signals indicative of a temperature of portions of the fluid sample in the microfluidic channel 1136. In the example illustrated, each of temperature sensors 1135 is located external to channel 1136 to indirectly sense a temperature of the sample fluid within channel 1136. In other implementations, temperature sensors 1175 are located within microfluidic reservoir 1134 to directly sense a temperature of the sample fluid within reservoir 1134. In yet another implementation, temperature sensors 1175 are located within channel 1136. In yet other implementations, temperature sensors 1175 may be located at other locations, wherein the temperature at such other locations is correlated to the temperature of the sample fluid being tested. In one implementation, temperature sensors 1135 output signals which are aggregated and statistically analyzed as a group to identify statistical value for the temperature of the sample fluid being tested, such as an average temperature of the sample fluid being tested. In one implementation, chip 1130 comprises multiple temperature sensors 1175 within reservoir 1134, multiple temperature sensors 1175 within channel 1136 and/or multiple temperature sensors external to the fluid receiving volume provided by reservoir 1134 and channel 1136, within the substrate of chip 1130.

In one implementation, each of temperature sensors 1175 comprises an electrical resistance temperature sensor, wherein the resistance of the sensor varies in response to changes in temperature such that signals indicating the current electrical resistance of the sensor also indicate or correspond to a current temperature of the adjacent environment. In other implementations, sensors 1175 comprise other types of micro-fabricated or microscopic temperature sensing devices.

Electrical contact pads 1177 are located on end portions of microfluidic chip 1130, which are spaced from one another by less than 3 mm and nominally less than 2 mm, providing microfluidic chip 1130 with a compact length facilitates the compact size of cassette 1110. Electrical contact pads 1177 sandwich the microfluidic sensing regions 1135 and are electrically connected to sensors 1152, 1154, pumps 1160 and temperature sensors 1175 by multiplexer circuitry 1179. Electrical contact pads 1177 are further electrically connected to the electrical connectors 1016 of cassette board 1112 (shown in FIG. 8).

Multiplexer circuitry 1179 is electrically coupled between electrical contact pads 1177 and sensors 1150, 1152, pumps 1160 and temperature sensors 1175. Multiplexer circuitry 1179 facilitates control and/or communication with a number of sensors 1138, pumps 1160 and temperature sensors 1175 that is greater than the number of individual electrical contact pads 1177 on chip 430.

For example, despite chip 1130 having a number n of contact pads, communication is available with a number of different independent components having a number greater than n. As a result, valuable space or real estate is conserved, facilitating a reduction in size of chip 1130 and cassette 1110 in which chip 1130 is utilized.

Multiplexer circuitry 1179 is similar to multiplexer 40 described above in that multiplexer circuitry 1179 combines signals from sensors 1150, 1152 and 1175 as a single data stream which is communicated across a single contact pad 1177. In one implementation, multiplexer circuitry 1179 may output a single data stream comprising all of the signals from all of the sensors, sensors 1150, 1152 and 1175 in an individual sensing region 1135 across a single contact pad 1177. In another implementation, multiplexer circuitry 1179 may output a single data stream comprising all those signals from all of the sensors of all of the different sensing regions 1135 across a single contact pad 1177. In still other implementations, multiplexer circuitry 1179 may output different streams of data, wherein each of the streams comprise signals from multiple sets of different sensors, sensors 1150, 1150 to 1175, of microfluidic chip 1130. As a result, the remaining contact pads may be utilized for control of other components of microfluidic chip 1130.

Although microfluidic chip 1130 is illustrated as comprising three sensors that sense different physical properties of fluid, in other implementations, microfluidic chip 1130 may comprise other sensors that sense other physical properties of fluid. For example, in other implementations, microfluidic chip 1130 may comprise a pressure sensor. In some implementations, microfluidic chip 1130 may comprise additional impedance, optical and/or temperature sensors at other locations within each sensing region 1135. In some implementations, particular sensors may be located off-chip, wherein the data from the sensor is transmitted to the single data stream. For example, one of the sensors may comprise an optical external camera that captures specific portions of the chip, wherein the images are sent to an FPGA on the chip through a connector, such as a universal serial bus connection.

Referring back to FIG. 8, cassette interface 1200, sometimes referred to as a “reader” or “dongle”, may interconnect and serve as an interface between cassette 1110 and mobile analyzer 1232. Cassette interface 1200 contains components or circuitry that is dedicated, customized or specifically adapted for controlling components of microfluidic cassette 1110. Cassette interface 220 carries circuitry and electronic components dedicated or customized for the specific use of controlling the electronic components of cassette 1110. Because cassette interface 1200 carries much of the electronic circuitry and components specifically dedicated for controlling the electronic components of cassette 1110 rather than such electronic components being carried by cassette 1110 itself, cassette 1110 may be manufactured with fewer electronic components, allowing the costs, complexity and size of cassette 1110 to be reduced. As a result, cassette 1110 is more readily disposable after use due to its lower base cost. Likewise, because cassette interface 1200 is releasably connected to cassette 1110, cassette interface 1200 is reusable with multiple exchanged cassettes 1110. The electronic components carried by cassette interface 1200 and dedicated or customized to the specific use of controlling the electronic components of a particular cassette 1110 are reusable with each of the different cassettes 1110 when performing fluid or blood tests on different fluid samples or fluid samples from different patients or sample donors.

In the example illustrated, cassette interface 1200 comprises electrical connector 1204, electrical connector 1206 and firmware 1208 (schematically illustrated external to the outer housing of interface 1200). Electrical connector 1204 comprises a device by which cassette interface 1200 is releasably electrically connected directly to electrical connectors 1116 of cassette 1110. In one implementation, the electrical connection provided by electrical connector 1204 facilitates transmission of electrical power for powering electronic components of microfluidic chip 1130, such as sensors 1152, 1154 or a microfluidic pump 1160. In one implementation, the electrical connection provided by electrical connector 1204 facilitates transmission of electrical power in the form of electrical signals providing data transmission to microfluidic chip 1130 to facilitate control of components of microfluidic chip 1130. In one implementation, the electrical connection provided by electrical connector 1204 facilitates transmission of electrical power in the form electrical signals to facilitate the transmission of data from microfluidic chip 1130 to the mobile analyzer 1232, such as the transmission of signals from sensor sensors 38. In one implementation, electrical connector 1204 facilitates each of the powering of microfluidic chip 1130 as well as the transmission of data signals to and from microfluidic chip 1130.

In the example illustrated, electrical connectors 1204 comprise a plurality of electrical contact pads located in a female port, wherein the electrical contact pads which make contact with corresponding pads 1116 of cassette 1110. In yet another implementation, electrical connectors 1204 comprise a plurality of electrical prongs or pins, a plurality of electrical pin or prong receptacles, or a combination of both. In one implementation, electrical connector 1204 comprises a universal serial bus (USB) connector port to receive one end of a USB connector cord, wherein the other end of the USB connector cord is connected to cassette 1110. In still other implementations, electrical connector 1204 may be omitted, where cassette interface 1200 comprises a wireless communication device, such as infrared, RF, Bluetooth other wireless technologies for wirelessly communicating between interface 1200 and cassette 1110.

Electrical connector 1204 facilitates releasable electrical connection of cassette interface 1200 to cassette 1110 such that cassette interface 1200 may be separated from cassette 1110, facilitating use of cassette interface 1200 with multiple interchangeable cassettes 1110 as well as disposal or storage of the microfluidic cassette 1110 with the analyzed fluid, such as blood. Electrical connectors 1204 facilitate modularization, allowing cassette interface 1200 and associated circuitry to be repeatedly reused while cassette 1110 is separated for storage or disposal.

Electrical connector 1206 facilitates releasable connection of cassette interface 1200 to mobile analyzer 1232. As a result, electrical connector 1206 facilitates use of cassette interface 1200 with multiple different portable electronic devices 1232. In the example illustrated, electrical connector 1206 comprises a universal serial bus (USB) connector port to receive one end of a USB connector cord 1209, wherein the other end of the USB connector cord 1209 is connected to the mobile analyzer 1232. In other implementations, electrical connector 1206 comprises a plurality of distinct electrical contact pads which make contact with corresponding blood connectors of mobile analyzer 1232, such as where one of interface 1200 and mobile analyzer 1232 directly plug into the other of interface 1200 and mobile analyzer 1232. In another implementation, electrical connector 1206 comprises prongs or prong receiving receptacles. In still other implementations, electrical connector 1206 may be omitted, where cassette interface 1200 comprises a wireless communication device, utilizing infrared, RF, Bluetooth or other wireless technologies for wirelessly communicating between interface 1200 and mobile analyzer 1232.

Firmware 1208 comprises electronic componentry and circuitry carried by cassette interface 1200 and specifically dedicated to the control of the electronic components and circuitry of microfluidic chip 1130 and cassette 1110. In the example illustrated, firmware 1208 serves as part of a controller to control sensors 1150, 1152. In implementations where firmware 1208 comprises a field programmable gate array or an ASIC, the field programmable gate array or ASIC may additionally serve as a driver for other electronic components on microfluidic chip 1130 such as microfluidic pumps 1160 (such as resistors), temperature sensors 1175 and other electronic components upon the microfluidic chip.

Mobile analyzer 1232 comprises a mobile or portable electronic device to receive data from cassette 1110. Mobile analyzer 1232 is releasably or removably connected to cassette 1110 indirectly via cassette interface 1200. Mobile analyzer 1232 performs varies functions using data received from cassette 1110. For example, in one implementation, mobile analyzer 1232 stores the data. In the example illustrated, mobile analyzer 1232 additionally manipulates or processes the data, displays the data and transmits the data across a local area network or wide area network (network 1500) to a remote analyzer 1300 providing additional storage and processing.

In the example illustrated, mobile analyzer 1232 comprises electrical connector 1502, power source 1504, display 1506, input 1508, processor 1510, and memory 1512. In the example illustrated, electrical connector 1502 is similar to electrical connectors 1206. In the example illustrated, electrical connector 1502 comprises a universal serial bus (USB) connector port to receive one end of a USB connector cord 1209, wherein the other end of the USB connector cord 1209 is connected to the cassette interface 1200. In other implementations, electrical connector 1502 comprises a plurality of distinct electrical contact pads which make contact with corresponding electrical connectors of interface 1200, such as where one of interface 1200 and mobile analyzer 1232 directly plug into the other of interface 1200 and mobile analyzer 1232. In another implementation, electrical connector 1206 comprises prongs or prong receiving receptacles. In still other implementations, electrical connector 1502 may be omitted, where mobile analyzer 1232 and cassette interface 1200 each comprise a wireless communication device, utilizing infrared, RF, Bluetooth or other wireless technologies for facilitating wireless communication between interface 1200 and mobile analyzer 1232.

Power source 1504 comprises a source of electrical power carried by mobile analyzer 1232 for supplying power to cassette interface 1200 and cassette 1110. Power source 1504 comprises various power control electronic componentry which control characteristics of the power (voltage, current) being supplied to the various electronic components of cassette interface 1200 and cassette 1110. Because power for both cassette interface 1200 and cassette 1110 are supplied by mobile analyzer 1232, the size, cost and complexity of cassette interface 1200 and cassette 1110 are reduced. In other implementations, power for cassette 1110 and cassette interface 1200 are supplied by a battery located on cassette interface 1200. In yet another implementation, power for cassette 1110 is provided by a battery carried by cassette 1110 and power for interface 1200 is supplied by a separate dedicated battery for cassette interface 1200.

Display 1506 comprises a monitor or screen by which data is visually presented. In one implementation, display 1506 facilitates a presentation of graphical plots based upon data received from cassette 1110. In some implementations, display 1506 may be omitted or may be replaced with other data communication elements such as light emitting diodes, auditory devices are or other elements that indicate results based upon signals or data received from cassette 1110.

Input 1508 comprises a user interface by which a person may input commands, selection or data to mobile analyzer 1232. In the example illustrated, input 1508 comprise a touch screen provided on display 1506. In one implementation, input 1508 may additionally or alternatively utilize other input devices including, but are not limited to, a keyboard, toggle switch, push button, slider bar, a touchpad, a mouse, a microphone with associated speech recognition machine-readable instructions and the like. In one implementation, input 1506 facilitates input of different fluid tests or modes of a particular fluid test pursuant to prompts provided by an application program run on mobile analyzer 1232.

Processor 1510 comprises at least one processing unit to generate control signals controlling the operation of sensors 1138 as well as the acquisition of data from sensors 1138. Processor 1510 further outputs control signals controlling the operation of pumps 1160 and temperature sensors 1175. In the example illustrated, processor 1510 further analyzes data received from chip 1130 to generate output that is stored in memory 1512, displayed on display 1506 and/or further transmitted across network 1500 to remote analyzer 1300.

Memory 1512 comprises a non-transitory computer-readable medium containing instructions for directing the operation of processor 1510. As schematically shown by FIG. 6, memory 1512 comprises or stores data identification and routing instructions (DI) 1518, an application programming interface 1520 and application program 1522.

Data identification and routing instructions 1518 comprises structure for directing processor 1510 to discern, distinguish and identify (A) data and signals originating from or based upon signals output by sensor 1150 in the single data stream, (B) data and signals originating from or based upon signals output by sensor 1152 in the single data stream and (C) data and signals originating from or based upon signals output by sensor 1175 in the single data stream. In one implementation, data identifier instructions 1518 direct processor 1510 to read data bits contained in a header associated with each set or group of data bits from sensors 1150, 1152 and 1175 which are part of the single data stream from microfluidic chip 1130. Data identification and routing instructions 1518 direct processor 1510 to route the signals and/or data originating from or based upon signals the different sensors to different buffers or queue for subsequent processing by application program 1522 utilizing application programming interface 1520.

Application programming interface 1520 comprises a library of routines, protocols and tools, which serve as building blocks, for carrying out various functions or tests using cassette 1110. Application programming interface 1520 comprises programmed logic that accesses the library and assembles the “building blocks” or modules to perform a selected one of various functions or tests using cassette 1110. For example, in one implementation, application programming interface 1520 comprises an application programming interface library that contains routines for directing the firmware 1208 to place sensors 1150, 1152 in selected operational states. In the example illustrated, the library also contains routines for directing firmware 1208 to operate fluid pumps 1160 or dynamically adjusts operation of such pumps 1160 or sensors 1152, 1154 in response to a sensed temperature of the fluid being tested from temperature sensors 1175. In one implementation, mobile analyzer 1232 comprises a plurality of application programming interfaces 1520, each application programming interface 1520 being specifically designed are dedicated to a particular overall fluid or analyte test. For example, one application programming interface 1520 may be directed to performing cytology tests. Another application program interface 1520 may be directed to performing coagulation tests. In such implementations, the multiple application programming interfaces 1520 may share the library of routines, protocols and tools.

Application programming interface 1520 facilitates testing of fluids using cassette 1110 under the direction of different application programs. In other words, application programming interface 1520 provides a universal programming or software set of commands for firmware 1208 that may be used by any of a variety of different application programs. For example, a user of mobile analyzer 1232 is able to download or install any of a number of different application programs, wherein each of the different application programs is designed to utilize the application program interface 1520 so as to carry out tests using cassette 1110. As noted above, firmware 1208 interfaces between application programming interface 1520 and the actual hardware or electronic componentry found on the cassette 1110 and, in particular, microfluidic chip 1130.

Application program 1522 comprises an overarching machine-readable instructions contained in memory 1512 that facilitates user interaction with application programming interface 1520 or the multiple application programming interfaces 1520 stored in memory 1512. Application program 1522 presents output on display 1506 and receives input through input 1508. Application program 1522 communicates with application program interface 1520 in response to input received through input 1508. For example, in one implementation, a particular application program 1522 presents graphical user interfaces on display 1506 prompting a user to select which of a variety of different testing options are to be run using cassette 1110. Based upon the selection, application program 1522 interacts with a selected one of the application programming interfaces 1520 to direct firmware 1208 to carry out the selected testing operation using the electronic componentry of cassette 1110. Sensed values received from cassette 1110 using the selected testing operation are received by firmware 1208 and are processed by the selected application program interface 1520. The output of the application programming interface 1520 is generic data, data that is formatted so as to be usable by any of a variety of different application programs. Application program 1522 presents the base generic data and/or performs additional manipulation or processing of the base data to present final output to the user on display 1506.

Although application programming interface 1520 is illustrated as being stored in memory 1512 along with the application program 1522, in some implementations, application programming interface 1520 is stored on a remote server or a remote computing device, wherein the application program 1522 on the mobile analyzer 1232 accesses the remote application programming interface 1520 across a local area network or a wide area network (network 1500). In some implementations, application programming interface 1520 is stored locally on memory 1512 while application program 1522 is remotely stored a remote server, such as server 1300, and accessed across a local area network or wide area network, such as network 1500. In still other implementations, both application programming interface 1520 and application program 1522 are contained on a remote server or remote computing device and accessed across a local area network or wide area network (sometimes referred to as cloud computing).

FIG. 14 is a diagram illustrating one example process of data handling that may be carried out by system 1000. As schematically illustrated, chip 1130 outputs a single data stream 1542 which includes data or signals from each of sensors 1150, 1152 and 1175. Processor 1510 of mobile analyzer 1232 receives data thread 1542. Following the data identification and routing instructions 1518, processor 1510 identifies and distinguishes the impedance data signals from sensor 1150, the optical data signals from sensor 1152 and the temperature or thermal data signals from sensor 1175. As shown by FIG. 14, the impedance data signals from a sensor 1150 are routed to a queue or buffer for subsequent processing by an impedance data processing thread 1560 carried out by processor 1510 as the thread 1560 becomes free pursuant to instructions provided by application program 1522 using application programming interface 1520. The optical data signals from a sensor 1152 are routed to a queue or buffer for subsequent processing by an optical data processing thread 1562 carried out by processor 1510 as the thread 1562 becomes free pursuant to instructions provided by application program 1522 using application programming interface 1520. Likewise, the optical data signals from a sensor 1175 are routed to a queu or buffer for subsequent processing by a thermal data processing thread 1564 carried out by processor 1510 as the thread 1564 becomes free pursuant to instructions provided by application program 1522 using application programming interface 1520.

The three different types of data processing threads, impedance, optical and thermal, are concurrently carried out by processor 1510. As the data is processed, processor 1510 continuously, in real-time, outputs the results on display 1506, results R1 from thread 1560, results R2 from thread 1562 and results R3 from thread 1564. In some implementations, the results may comprise plotted graphs, wherein the graphs are continuously updated as new data is processed by the different threads.

Although the present disclosure has been described with reference to example implementations, workers skilled in the art will recognize that changes may be made in form and detail without departing from the spirit and scope of the claimed subject matter. For example, although different example implementations may have been described as including one or more features providing one or more benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following claims is manifestly intended to be as broad as possible. For particular element also encompass a plurality of such particular elements. The terms “first”, “second”, “third” and so on in the claims merely distinguish different elements and, unless otherwise stated, are not to be specifically associated with a particular order or particular numbering of elements in the disclosure.

What is claimed is:

1. A method for handling multimode microfluidic data, the method comprising:
sensing a fluid within a microfluidic channel with a first sensor on a microfluidic chip and outputting first signals from the first sensor;
sensing the fluid within the microfluidic channel with a second sensor on the microfluidic chip and outputting second signals from the second sensor;
routing the first signals and the second signals as a single data stream.

2. The method of claim 1 further comprising
receiving the single data stream from the microfluidic chip;
routing the first signals from the first sensor to a first data processing thread; and
routing the second signals from the second sensor to a second data processing thread.

3. The method of claim 1, wherein the first signals comprise signals from an electrical sensor and wherein the second signals comprise signals from an optical sensor.

4. The method of claim 1, wherein the first signals comprise signals representing impedance data and wherein the second signals comprise signals representing thermal data.

5. The method of claim 2, wherein the single data stream comprises third signals from a third sensor, the method further comprising processing the third signals from the third sensor with a third data processing thread.

6. The method of claim 5, wherein the first signals comprise signals representing impedance data, wherein the second signals comprise signals representing thermal data and wherein the third signals comprise signals representing optical data.

7. The method of claim 1 further comprising outputting the first signals at a first frequency and outputting the second signals at a second frequency different than the first frequency.

8. The method of claim 7, wherein the first signals represent the electrical impedance data and wherein the second signals represent thermal data and wherein the first frequency is greater than the second frequency.

9. The method of claim 7, wherein the first signals represent impedance data and with the second signals represent optical data and wherein the first frequency is greater than the second frequency.

10. The method of claim 2 further comprising:
processing the first signals with the first data processing thread as part of a first sample test;
processing the second signals with the second data processing thread as part of a second sample test; and
concurrently displaying results of the first sample test and the second sample test.

11. An apparatus comprising:
a non-transitory computer-readable medium containing instructions to direct a processor to:
identify a first type of data represented by a first signal in a single data stream of multimode data received from a microfluidic chip and route the first signal to a first data processing thread;
identify a second type of data, different than the first type of data, represented by a second signal in the single data stream of multimode data received from the microfluidic chip and route the second signal to a second data processing thread; and
concurrently output, in real time, results of the first data processing thread and the second data processing thread.

12. The apparatus of claim 11, wherein the instructions are to further direct the processor to:
identify the third type of data, represented by a third signal in the single data stream from the microfluidic chip;
route the third signal to a third data processing thread; and
concurrently output, in real time, results of the third processing thread with the results of the first processing thread and the second processing thread.

13. The apparatus of claim 12, wherein first type of data comprises electrical impedance data, wherein the second type of data comprises optical data and wherein the third type of data comprises thermal data.

14. An apparatus comprising:
a microfluidic chip to sense multimode data, the microfluidic chip comprising:
a microfluidic channel to receive a fluid;
a first sensor to sense and output first signals indicating a first fluid characteristic;
a second sensor to sense and output second signals indicating a second fluid characteristic; and
an integrated circuit to route the first signals and the second signals as a single data stream.

15. The apparatus of claim 14 further comprising:
a mobile analyzer comprising:
a processor;
a non-transitory computer-readable medium containing instructions to direct the processor to:
identify the first signals in the single data stream received from the microfluidic chip and route the first signals to a first data processing thread; and
identify the second signals in the single data stream received from the microfluidic chip and route the second signals to a second data processing thread.
